(19) United States
(12) Patent Application Publication
(10) Pub. No.: US 2011/0129910 A1
(43) Pub. Date: Jun. 2, 2011

(54) COOLING DEVICE, AND ASSEMBLY, AND METHODS FOR LOWERING TEMPERATURE IN A CHEMICAL REACTION

(75) Inventors: Tae Goo Kang, Singapore (SG); Cheng Fang, (US); Siow Pin Melvin Tan, Singapore (SG); Hongmiao Ji, Singapore (SG); Xiaowu Zhang, Singapore (SG); Yu Chen, Singapore (SG)

(73) Assignee: Agency for Science, Technology and Research

(21) Appl. No.: 12/907,214

(22) Filed: Oct. 19, 2010

(30) Foreign Application Priority Data
Oct. 20, 2009 (SG) 200907005-3

(51) Int. Cl. C12M 1/00; F25D 5/00

(52) U.S. Cl. 435/289.1; 62/4

(57) ABSTRACT

Embodiments provide a cooling device. The cooling device comprises a container configured as a heat sink. The container is at least partially made from heat conducting material. The cooling device further comprises endothermic chemical material which is contained in the container.

The present application claims the benefit of the Singapore provisional application 200907005-3 (filed on 20 Oct. 2009), the entire contents of which are incorporated herein by reference for all purposes.

### TECHNICAL FIELD

Embodiments relate generally to a cooling device, an assembly, and methods for lowering the temperature of a chemical reaction which is carried out in a chemical reaction module or in a chemical reaction chip.

### BACKGROUND

Recently, biomolecular techniques have enabled faster diagnosis of infectious diseases such as Dengue and H1N1 by directly detecting the viral genome (e.g. viral ribonucleic acid (RNA)) in the blood of patients using polymerase chain reaction (PCR), with or without reverse transcription (RT-PCR). PCR can be used to amplify a specific region of a nucleic acid strand. For example, PCR may consist of 20-40 cycles of temperature changes during which thousands to millions of copies of a particular deoxyribonucleic acid (DNA) sequence can be generated. For another example, reverse transcription-PCR (RT-PCR) amplifies viral RNA by enzymatic reaction and thermal cycling.

However, the conventional technique of PCR generally requires sample preparation steps, such as cell lysis, viral genome (e.g. RNA) extraction and its purification which needs experienced personnel and well-equipped laboratory facilities. The need for experienced personnel and well-equipped laboratory facilities prevents the analysis to be performed near the patient and leads to limited time-to-results, which is critical for early diagnostics and disease outbreak control. Advanced technologies for point-of-care (POC) systems have been highlighted due to their potential to remove the labor-intensive, time-consuming and high-cost processes from the laboratory and bring it to the patient. In this context, the POC systems refer to the system for testing or diagnosis near the site of patient care.

Recent technological advances have enabled automation and miniaturization of some of the steps, such as viral RNA extraction, microchip scale RNA/DNA amplification process by PCR, detection process of amplified PCR product, etc. Among those steps, hybridization based detection method such as silicon nanowire biosensor or microarray chip has been popular due to its high performance, reliability and manufacturability. The hybridization based detection method typically requires the nucleic acid, e.g. DNA, to be in a single stranded form rather than double stranded form. For example, according to the hybridization based detection method, the single stranded DNA may bind with a pre-treated nanowire, thereby causing the change of the resistance of nanowire. Accordingly, detection of a change of the resistance of the nanowire may indicate the existence of the DNA. Thus, after the PCR amplification process, the DNA is required to be denatured and remained in a single stranded form for hybridization based detection. For a full sample-to-answer microsystem for nucleic acid detection based on DNA/PNA hybridization (e.g. with a nanowire biosensor), an additional super-cooling module is essential for denaturing the PCR amplicon after amplification.

FIG. 1 (a) shows an example of the temperature changes over time during the process of a reverse transcription PCR (RT-PCR) and DNA denaturation. DNA strands are amplified during the PCR thermal cycling period 101. After the PCT thermal cycling period 101, the DNA is denatured during the denaturation period 102. During the denaturation period 102, the DNA is heated up to a denaturation temperature such that two strands of each DNA double helix are separated. Then a super cooling is applied in order to prevent the amplified DNA to hybridize in a double strand form. In this context, super cooling refers to cooling down of the PCR product from denaturation temperature to under the room temperature rapidly after PCR cycling but before hybridization process. The room temperature refers to temperature ranging from 20° C. to 25° C., for example. Denaturation of DNA after the PCR thermal cycling is preferred in circumstances, for example, when double stranded DNA can not be detected or can not be easily detected by a sensor for detecting the PCR product while single stranded DNA can be detected. For example, the denaturation process as shown in denaturation period 102 is preferably applied for hybridization based detection.

FIG. 1 (b) shows an enlarged picture of the temperature changes during a single cycle of the PCR thermal cycling period 101. Each cycle generally includes a denaturation period 103, an annealing period 104, and an extension period 105. In the denaturation period 103, two strands in a DNA double helix are separated at denaturation temperature, e.g. 95° C. Then the temperature is lowered to an annealing temperature, e.g. 55° C., and the annealing period 104 begins. During the annealing period, primers are annealed to single stranded DNA template. Then the temperature is further increased to an extension temperature, e.g. 72° C., and the extension period 105 begins. During the extension period 105, a new DNA strand complementary to the DNA template strand is synthesized. Then the temperature is further increased to the denaturation temperature and another cycle begins.

Currently, microPCR system (or conventional PCR machine) adapts a heat-sink module for rapidly cooling down its PCR chamber temperature from the denaturation temperature (e.g. around 95° C.) to the annealing temperature (e.g. around 55˜60° C.). In this context, the heat sink generally refers to a component or assembly that transfers heat generated within a solid material to a fluid medium, such as air or a liquid. However, in order to realize a fully integrated microsystem including nucleic acid (e.g. DNA) hybridization detection, nucleic acid denaturation (e.g. DNA denaturation) by a super-cooling process which lowers temperature of the PCR product from the denaturation temperature to a temperature below room temperature in a fast manner is essential in addition to the heat sink function of PCR thermal cycling process which lowers temperature from the denaturation temperature to the annealing temperature. The super cooling may enable to prevent the amplified nucleic acid to hybridize in a double strand form that would not be detected by the sensor using hybridization based detection method.

Thus, two different cooling stages are preferably needed for a microsystem including nucleic acid (e.g. DNA) hybridization based detection to realize the fully integrated microsystem including micro polymerase chain reaction (PCR) with hybridization-based DNA detection module.

That is, one is targeting above room temperature (e.g. around 55° C.), and thus natural convection may be enough. The first cooling stage is for speeding up the PCR thermal cycling, and to achieve the cooling of the PCR chamber by lowering temperature from a denaturation temperature (e.g. around 95° C.) to an annealing temperature (e.g. around 55° C.). A heat sink may be used in the first cooling stage. The first step cooling is necessary during RT-PCR thermal cycling

The second cooling stage is targeting below room temperature (e.g. less than 10° C. in 10 seconds), and thus natural convection cooling may not be enough. The second step cooling is necessary, for example, for DNA denaturation after the RT-PCR thermal cycling in order to get single stranded DNA from the PCR product. Thus, there is time difference between those two different cooling stages. The second cooling stage is super-cooling. That is, after DNA is denatured at the denaturation temperature (e.g. around 95° C.), the temperature of the PRC product is dropped to a temperature below room temperature (e.g. less than 10° C.) rapidly after PCR cycling but before hybridization process, thereby remaining the DNA in a single stranded form for later hybridization-based DNA detection.

The major difference between these two cooling functions is the target temperature, one of which is above room temperature so that conventional natural convection or forced convection method may be used, and the other one of which is lower than room temperature so that natural convection or forced convection method may not be enough to get the target temperature.

The conventional way for the first cooling function (heat-sink) uses a metallic fin structure having good thermal conduction and high surface-to-volume ratio, or a metallic fin structure with extra fan to improve the convection efficiency. However, the convection-based cooling method can not cool down the temperature below the room temperature. For conventional PCR machine as well as most of the chip cooling cases, only the first stage of cooling is needed, and any shape of the metallic structure may work. Especially fin-shaped structure is known to be the best solution. However, in circumstances such as hybridization based detection is needed, the second stage of cooling as described herein is required.

The conventional way for reaching below room temperature includes thermoelectric cooling (TEC) devices, e.g. Peltier effect or Joule-Thomson (JT) refrigerator devices, using sudden expansion of refrigerant through the capillary tube. Both methods are well-known for IC chip cooling to prevent overheating and decreased performance and durability. However, the relatively slow transient response of both methods due to their high loading effect makes them unfit for the super-cooling required for the second stage cooling described here. Moreover, it is difficult to integrate the TEC cooling devices which requires the consumption of substantial electrical power within a fluidic microsystem.

Thus, there is need to develop novel cooling device which has a two-stage cooling function for application such as for nucleic aid based infectious disease diagnostics tools and which is suitable to be integrated within a fluidic microsystem.

### SUMMARY OF THE INVENTION

Various embodiments provide a cooling device which may provide two different levels of cooling and which is suitable to be integrated into a microsystem, for example, for nucleic aid based infectious disease diagnostics.

In one embodiment, the cooling device may include a container. The container may be configured as a heat sink. In one embodiment, the container is at least partially made from heat conducting material. In one embodiment, the cooling device further includes endothermic chemical material. The endothermic chemical material may be contained in the container.

In one embodiment, an assembly is provided. The assembly may include a module and a cooling device. The module may be configured to perform a chemical reaction. The module may include a supporting element and a chemical reaction chip coupled to the supporting element. In one embodiment, the chemical reaction is carried out in the chemical reaction chip. The cooling device may be in accordance with the cooling device as described herein to cool at least a part of the chemical reaction chip.

In one embodiment, a method for lowering the temperature of a chemical reaction which is carried out in a chemical reaction chip integrated into a chemical reaction module is provided. In one embodiment, a cooling device as described herein may be used. In one embodiment, the method may include cooling the chemical reaction chip to a first temperature using the cooling device. In one embodiment, the method may further include cooling the chemical reaction chip to a second temperature using the cooling device by initializing an endothermic reaction of the endothermic chemical material in the cooling device.

It should be noted that the embodiments describing the cooling device are also analogously valid for the corresponding assembly and method where applicable. It should also be noted that embodiments describing the assembly are also analogously valid for the corresponding method where applicable.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 (a) shows an example of the temperature change over time during the reverse-transcription polymerase chain reaction (RT-PCR) thermal cycling period and the denaturation of DNA;

FIG. 1 (b) shows an enlarged view of the temperature change for a single cycle of PCR thermal cycling;

FIG. 2 (a) shows a cooling device according to one embodiment;

FIG. 2 (b) shows a perspective bottom view of the cooling device in one embodiment;

FIG. 2 (c) shows the base element of the cooling device in one embodiment;

FIG. 3 (a) illustrates the assembly map of an assembly according to one embodiment;

FIG. 3 (b) illustrates an assembly according to one embodiment;

FIG. 3 (c) shows an example of the temperature changes of the PCR product in a DNA denaturation process;

FIG. 4 illustrates a method for lowering the temperature of a chemical reaction which is carried out in a chemical reaction chip integrated into a chemical reaction module in one embodiment;

FIG. 5 (a) illustrates the photo of a module for performing a PCR in one exemplary embodiment;

FIG. 5 (b) shows a photo of a chemical reaction chip in the module shown in FIG. 5 (a) in one exemplary embodiment;

FIG. 5 (c) shows a photo of a cooling device in one exemplary embodiment;

FIG. 5 (d) shows a photo of a temperature control device;

FIG. 5 (e) illustrates a screen shot of the monitoring of temperature control during the PCR thermal cycling process;

FIG. 6 shows the temperature change during the PCR thermal cycling using a cooling device according to one embodiment;

FIG. 7 shows the temperature change after the PCR product is heated to a denaturation temperature under different cooling conditions; and

FIG. 8 shows the detection of single stranded DNA on chip for hybridization on the nanowire array for different PCR product samples.

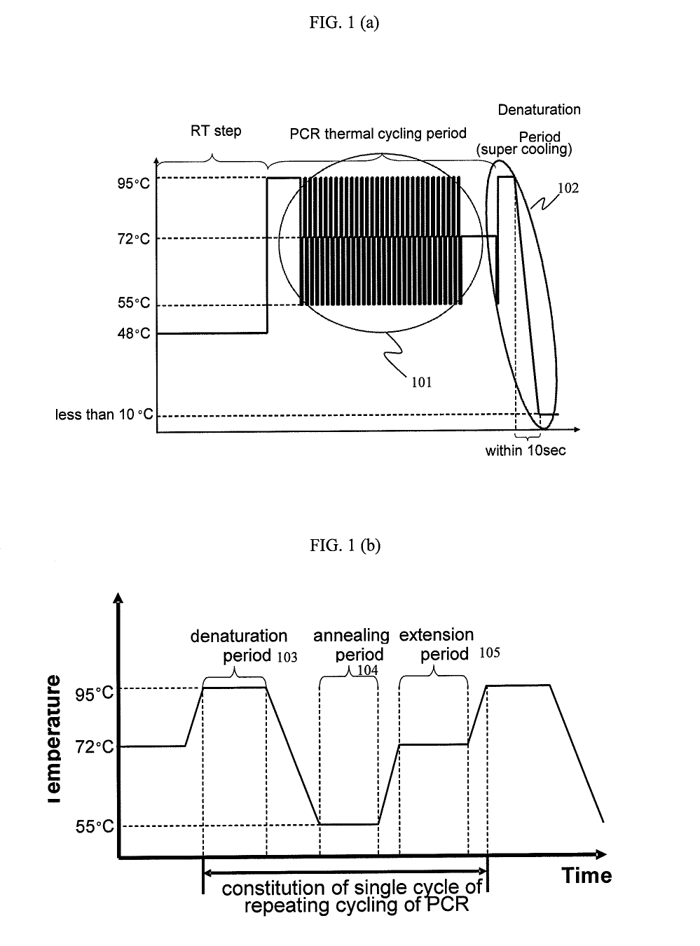

### DESCRIPTION

The following detailed description refers to the accompanying drawings that show, by way of illustration, specific details and embodiments in which the invention may be practiced. These embodiments are described in sufficient detail to enable those skilled in the art to practice the invention. In this regard, directional terminology, such as “top”, “bottom”, “front”, “back”, “leading”, “trailing”, etc, is used with reference to the orientation of the Figure(s) being described. Because components of embodiments can be positioned in a number of different orientations, the directional terminology is used for purposes of illustration and is in no way limiting. Other embodiments may be utilized and structural, logical, and electrical changes may be made without departing from the scope of the invention. The various embodiments are not necessarily mutually exclusive, as some embodiments can be combined with one or more other embodiments to form new embodiments. The following detailed description therefore, is not to be taken in a limiting sense, and the scope of the present invention is defined by the appended claims.

The word “exemplary” is used herein to mean “serving as an example, instance, or illustration”. Any embodiment or design described herein as “exemplary” is not necessarily to be construed as preferred or advantageous over other embodiments or designs.

Various embodiments provide a cooling device. The cooling device may include a container and endothermic chemical material. The container may be configured as a heat sink. The container may be at least partially made from heat conducting material. The endothermic chemical material is contained in the container. For example, the endothermic chemicals may be a mixture of urea and ammonium chloride, e.g. a mixture of 10 gram of urea and 30 gram of ammonium chloride.

In one embodiment, the container includes a base element and a covering element, which covers the base element. In one embodiment, the base element is a base chamber.

In one embodiment, the cooling device further includes a heat contact element on the outer surface of the base element. The heat contact element may be a heat contact plate.

In one embodiment, the base element is at least partially made from a heat conducting material. In one embodiment, the covering element is at least partially made from a heat conducting element.

In one embodiment, the heat contact element is at least partially made from a heat conducting material. In one embodiment, the heat contact element is at least partially made from metal. In one embodiment, the heat contact element is at least partially made from at least one material selected from a group of materials consisting of: gold, argent, aluminum, brass, zinc, magnesium, graphite, tungsten, silicon, molybdenum, nickel, iron, steel, platinum, tin, and tantalum.

In one embodiment, the base element is at least partially made from metal. In one embodiment, the base element is at least partially made from copper. In one embodiment, the base element is at least partially made from at least one material selected from a group of materials consisting of: gold, argent, aluminum, brass, zinc, magnesium, graphite, tungsten, silicon, molybdenum, nickel, iron, steel, platinum, tin, and tantalum.

In one embodiment, the covering element is at least partially made from metal. In one embodiment, the covering element is at least partially made from copper.

In one embodiment, the container includes at least one opening for injecting an initial start-up material into the container. In one embodiment, the at least one opening is arranged on the upper surface of the covering element.

It should be noted that the list of materials that may be used to make any part of the cooling device is not exhaustive. A skilled person would appreciate that any suitable material with suitable heat conducting characteristic may be selected for making at least a part of the cooling device.

In one embodiment, an assembly is provided. The assembly may include a module configured to perform a chemical reaction and a cooling device as described herein. The module may include a supporting element and a chemical reaction chip coupled to the supporting element. The chemical reaction may be carried out in the chemical reaction chip and the cooling device may be configured to cool at least a part of the chemical reaction chip. The supporting element may be a container.

In one embodiment, the chemical reaction is a biochemical reaction. In one embodiment, the chemical reaction is a polymerase chain reaction (PCR). In one embodiment, the chemical reaction chip is a polymerase chain reaction chip.

In one embodiment, the supporting element contains electrical interconnections and fluidic interconnections.

In one embodiment, the module further includes a top plate which covers the chemical reaction chip. In one embodiment, the top plate includes an opening. The heat contact element of the cooling device may be fed through the opening of the top plate. According to one embodiment, the heat contact element of the cooling device is fed through the opening of the top plate in such a manner that the heat contact element contacts top surface of the chemical reaction chip such that thermal conduction between the cooling device and the top surface of the chemical reaction chip is realized. The top plate may assist the positioning of the cooling device.

In one embodiment, the cooling device may be configured as a heat-sink for leading off heat from the module. In one embodiment, the chemical reaction is polymerase chain reaction and the cooling device is configured to additionally cool the module or the chemical reaction chip in the module by initializing an endothermic reaction of the endothermic chemical material. In one embodiment, the cooling device is configured to additionally cool the module by initializing an endothermic reaction of the endothermic chemical material using water as initial start-up.

According to one embodiment, the chemical reaction is polymerase chain reaction and the cooling device is configured to cool the module or the chemical reaction chip in a first stage in a polymerase chain reaction process from denaturation temperature to a first temperature, which is the annealing temperature, using the cooling device as a heat-sink, and to cool the module in a second stage from denaturation temperature to a second temperature by initializing an endothermic reaction of the endothermic chemical material.

In one embodiment, the first temperature is a temperature in the range from about 50 to about 60° C., and the second temperature is a temperature lower than room temperature, e.g. less than about 10° C. In one embodiment, the first temperature is approximately 55° C.

In one embodiment, the module configured to perform a polymerase chain reaction is configured to carry out polymerase chain reaction with reverse transcription. In another embodiment, the module configured to perform a polymerase chain reaction is configured to carry out polymerase chain reaction without reverse transcription.

In a further embodiment, the cooling device is configured to cool the module in a first stage to a first temperature, which is the annealing temperature, and the cooling device is configured as a heat-sink for thermal cycling in polymerase chain reaction. The cooling device is further configured to cool the module in a second stage to a second temperature by initializing an endothermic reaction of the endothermic chemical material for preventing nucleic acids from hybridization. The nucleic acids may be one of DNA, RNA and PNA.

According to one embodiment, a method for lowering the temperature of a chemical reaction which is carried out in a chemical reaction chip integrated into a chemical reaction module using the cooling device as described herein is provided. The method may include cooling the chemical reaction chip to a first temperature using the cooling device, and cooling the chemical reaction chip to a second temperature using the cooling device by initializing an endothermic reaction of the endothermic chemical material in the cooling device.

In one embodiment, the chemical reaction is a biochemical reaction. In a further embodiment, the chemical reaction is a polymerase chain reaction.

In one embodiment, the initialization of the endothermic reaction of the endothermic chemical material is carried out using a liquid as initial start-up. For example, the liquid is water.

In one embodiment, the first temperature is a temperature in the range from about 50 to 60° C., and the second temperature is a temperature less than 10° C. For example, the first temperature is a temperature of approximately 55° C. The second temperature may be a temperature below room temperature.

In one embodiment, the polymerase chain reaction is carried out with reverse transcription. In another embodiment, the polymerase chain reaction is carried out without reverse transcription.

In one embodiment, the second stage is carried out for a cooling time of less than 10 seconds.

In one embodiment, the chemical reaction is polymerase chain reaction and the first temperature is an annealing temperature.

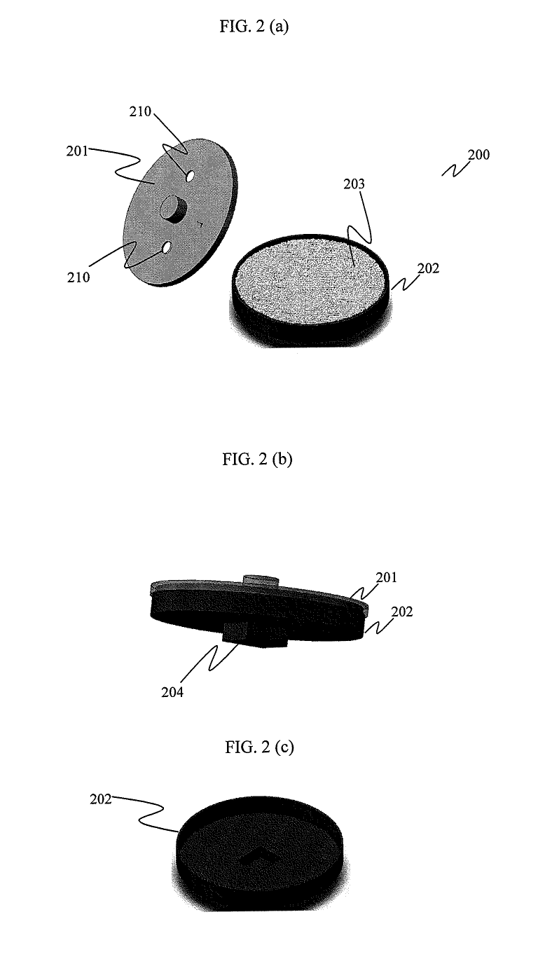

FIG. 2 (a) illustrates a cooling device 200 according to one exemplary embodiment. The cooling device 200 includes a container as a heat sink and endothermic material 203. The container may be at least partially made from heat conducting material. The endothermic chemical material 203 is contained in the container. The cooling device 200 may also be referred to as a two step passive cooling device as the cooling device 200 is able to provide two levels of cooling without the use of an external controlling device.

The container includes a base element 202 and a covering element 201, which covers the base element 202. The base element 202 may be a base chamber. It should be noted that the shape of the container is not restricted to the one as shown in FIG. 2 (a). A skilled person in the art would appreciate that theoretically any shape of container may be used. The selection of the shape of the container may be such that the container can contain a proper amount of endothermic material and has a large surface area to facilitate the dissipation of heat, for example.

FIG. 2 (b) illustrates a perspective bottom view of the cooling device 200 as shown in FIG. 2 (a). The cooling device 200 further includes a heat contact element 204 on the outer surface of the base element 202. The heat contact element 204 may be a heat contact plate. The heat contact plate may be of rectangular shape. For example, the heat contact element may be in contact with or be close to a chemical reaction chip when the cooling device 200 is in operation such that the cooling device can dissipate heat of the chemical reaction chip via the heat contact element 204, thereby lowering the temperature of the chemical reaction in the chemical reaction chip.

The base element 202 may be at least partially made from a heat conducting material. The covering element 201 may be at least partially made from a heat conducting element. The heat contact element 204 may be at least partially made from a heat conducting material such as metal. For example, the heat contact element 204 may be at least partially made from at least one material selected from a group of materials consisting of: gold, argent, aluminum, brass, zinc, magnesium, graphite, tungsten, silicon, molybdenum, nickel, iron, steel, platinum, tin, and tantalum.

The base element 202 may be at least partially made from metal, e.g. copper. For a further example, the base element 202 may be at least partially made from at least one material selected from a group of materials consisting of: gold, argent, aluminum, brass, zinc, magnesium, graphite, tungsten, silicon, molybdenum, nickel, iron, steel, platinum, tin, and tantalum. The covering element 201 may be at least partially made from metal, such as copper.

The container may includes at least one opening 210 for injecting an initial start-up material into the container. The at least one opening 210 may be arranged on the upper surface of the covering element. The initial start-up material may be water, for example. The initial start-up material may initiate a chemical reaction with the endothermic chemical material in the container 202 of the cooling device 200 upon which heat is absorbed. Consequently, the temperature of the chemical reaction in the chemical reaction chip may be lowered.

FIG. 2 (c) illustrates a top view of the base element 202 in one exemplary embodiment.

The working mechanism of the cooling device 200 is as follows.

Assuming a polymerase chain reaction (PCR) is carried out in a PCR chamber and the DNA inside the PCR chamber is amplified. As the container of the cooling device 200 is at least made of heat conducting material, e.g. metal, the container may be functioned as a heat sink. For example, during each cycle of the PRC thermal cycling period, a part of the cooling device 200, e.g. the heat contact element 204, may be in contact of a PCR chamber, and after the temperature of the PCR chamber is raised up to the denaturation temperature, the container of the cooling device 200 can lead the heat off the PCR chamber through the heat contact element 204 such that the temperature of the PCR chamber drops to the annealing temperature. Although the container of the cooling device 204 contains endothermic material, the container may dissipate the heat effectively if it is configured that the container has a proper thermal conductivity and large surface area such that the temperature of the PCR chamber can be lowered from the denaturation temperature to the annealing temperature within a proper time period in each PCR thermal cycling period. When the dimension of the PCR chamber is in a micrometer to millimeter level, this can be easily realized by selecting, for example, metal, as the heat conducting material for making the container.

After the PCR thermal cycling period, the PCR product is heated to a denaturation temperature such that the DNA is denatured and turned into a single stranded form. To maintain the DNA in the single stranded form, a super cooling process may be applied by adding an initial start-up material, e.g. water, into the container of the cooling device 200. The initial start-up material may react with the endothermic chemical material contained in the container of the cooling device 200 and during the reaction heat is absorbed in a fast manner such that the PCR product may be cooled from the denaturation temperature to a temperature lower than room temperature within 10 seconds.

The cooling device 200 may be incorporated into an assembly which includes a module for performing a chemical reaction, e.g. PCR.

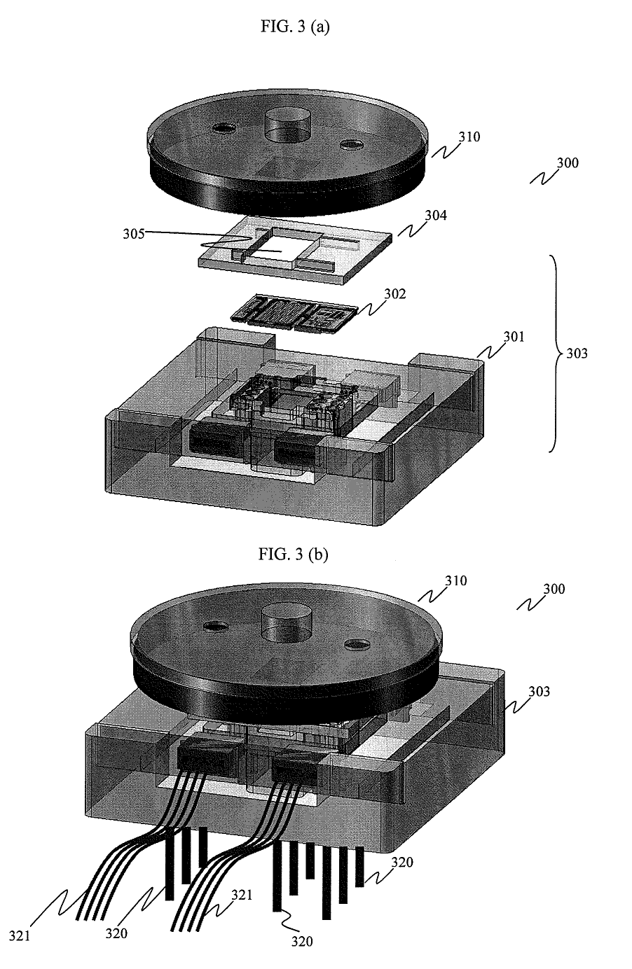

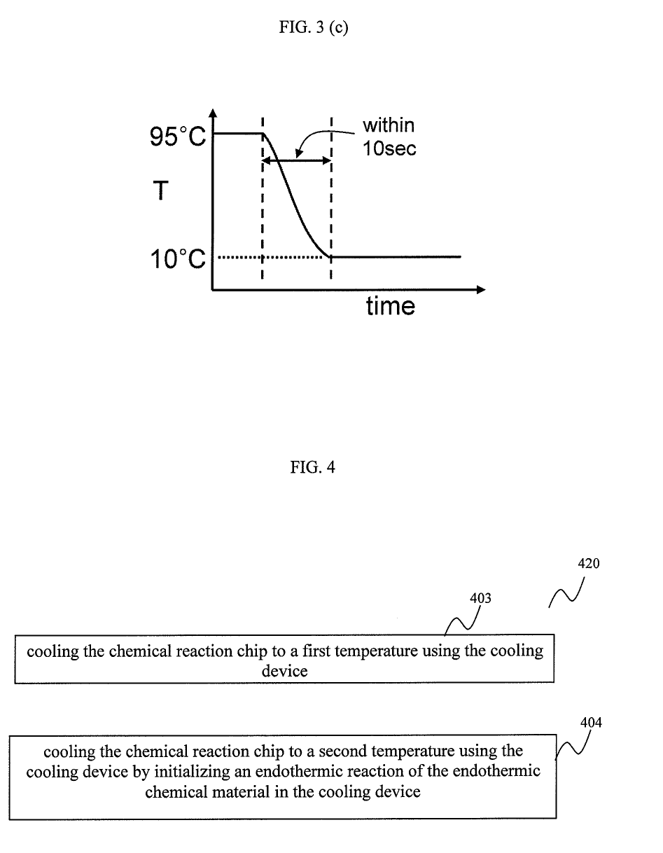

FIG. 3 (a) illustrates the assembly map of an assembly 300 according to one embodiment.

The assembly 300 may include a module 303 for performing a chemical reaction. The chemical reaction may be a biochemical reaction, e.g. a polymerase chain reaction. The assembly 300 may further include a cooling device 310. The cooling device 310 may be the same as the cooling device 200 as described with reference to FIGS. 2 (a)-(c). The cooling device 310 may be used to lower the temperature of the chemical reaction which is carried out in the module 303.

The module 303 may include a supporting element 301 and a chemical reaction chip 302 which is coupled to the supporting element 301. The supporting element 301 may be a container. The chemical reaction may be carried out in the chemical reaction chip 302. The chemical reaction chip 302 may be a polymerase chain reaction chip in which a polymerase chain reaction can be carried out. The cooling device 310 may be configured to cool at least a part of the chemical reaction chip 302.

The module 303 may be a microPCR module which includes the supporting element 301 having electrical interconnections as well as fluidic interconnections. The chemical reaction chip 302 may be a microPCR chip and may be placed on top of the supporting element 301. Optionally, the chemical reaction chip 302 may be covered by a top plate 304. In one exemplary embodiment, for the thermal isolation, normally top and bottom of the supporting element 301 have an opening at the place for the PCR chamber of the microPCR chip. Through this opening, the contact plate of cooling device 310 may be in contact with the PCR chamber to get the thermal conduction between heat container of the cooling device (e.g. metal chamber) and microPCR chamber of the chemical reaction chip 302.

The supporting element 301 of the module 303 may contain electrical interconnections and fluidic interconnections. The chemical reaction chip 302 may be in electrical connection and fluid connection with the supporting element 301.

The module 303 may further include a top plate 304 that covers the chemical reaction chip 302. The top plate may include an opening 305. The cooling device 310 may include a heat contact element which may be fed through the opening 305 of the top plate 304. The top plate 304 may assist the positioning of the cooling device 310. The heat contact element of the cooling device 310 may be fed though the opening of the top plate 304 in such a manner that the heat contact element of the cooling device 310 contact the module 303 such that thermal conduction between the cooling device 310 and the module 303, e.g. the chemical reaction chip 302, is realized. The cooling device 310 may be configured as a heat sink for leading off heat from the module 303. The cooling device 310 may be configured to additionally cool the module 303, e.g. the chemical reaction chip 302, by initializing an endothermic reaction of the endothermic chemical material contained in the cooling device 310. The endothermic reaction may be initiated using water as initial start-up.

In an alternative embodiment, the module 303 may not include a top plate. The cooling device 310 may be configured to be mounted on the module 303 such that both thermal isolation for the chemical reaction chip 302 and thermal conduction between the cooling device 310 and the module 303 may be achieved.

In one embodiment, the module 303 is configured to perform a polymerase chain reaction and the cooling device 310 is configured to cool the module 303 in a first stage in the polymerase chain reaction process from the denaturation temperature to a first temperature, which is the annealing temperature, using the cooling device 310 as a heat-sink. The cooling device 310 may be further configured to cool the module 303 in a second stage from denaturation temperature to a second temperature by initializing an endothermic reaction of the endothermic chemical material. The first temperature may be a temperature in the range from 50° C. to about 60° C., and the second temperature may be a temperature less than about 10° C. The first temperature may be about 55° C. The second temperature may be a temperature below room temperature.

The module 303 may be configured to perform a polymerase chain reaction with reverse transcription. Alternatively, the module 303 may be configured to perform a polymerase chain reaction without reverse transcription.

According to one embodiment, the module 303 is configured to perform a polymerase chain reaction, and the cooling device 310 is configured to cool at least a part of the module 303, e.g. the chemical reaction chip 302, in a first stage to a first temperature which is the annealing temperature where the cooling device 310 is further configured as a heat-sink for thermal cycling in polymerase chain reaction. The cooling device 310 may be further configured to cool at least a part of the module 303, e.g. the chemical reaction chip 302, in a second stage to a second temperature by initializing an endothermic reaction of the endothermic chemical material of r preventing nucleic acids from hybridization. In one embodiment, the nucleic acids are one of DNA, RNA, and PNA.

FIG. 3 (b) illustrates the assembly 300 (with microPCR module) according to one exemplary embodiment. In operation, the module 303 may be coupled with fluid supplying means 320. The module 303 may be further coupled electrical meaning 321.

The working principle of the assembly 300 is described as follows in one exemplary embodiment where the module 303 is configured to perform a polymerase chain reaction. It is however noted that the assembly 300 is not limited to be used for polymerase chain reaction but can be used for other reactions such as immunological reaction or those reactions that involves the temperature change and incubation.

Assuming the container of the cooling device 310 is a metallic chamber covered by a covering element, the endothermic chemical may be stored in powder form in the metallic chamber of the cooling device 310, and the chemical reaction chip 302 includes a microPCR chamber in which the polymerase chain reaction may be carried out. Before the PCR thermal cycling starts, the metallic chamber pre-contains the endothermic chemicals, covered by the covering element. The cooling device 310 may be placed on the microPCR chamber so that there will be thermal contact between microPCR chamber and heat contact plate of the cooling device 310. During the PCR thermal cycling, the metal chamber acts as a heat sink. At this stage, no water is added into the container of the cooling device and no endothermic reaction is carried out. Even though there are endothermic chemicals inside the chamber, the metal chamber may dissipate the heat effectively due to its high thermal conductivity and large surface area. After PCR thermal cycling, the PCR amplification product need to be heated up to the denaturation temperature (e.g. around 95° C.) and then rapidly cooled down to less than 10° C. within 10 seconds to get the single stranded DNA. For this to happen, water may be timely induced into the container of the cooling device 310 through the openings on the top element, thereby activating the endothermic chemical reaction. The pre-contained endothermic chemicals react with water such that heat is absorbed rapidly, and thus the temperature of the PCR chamber is lowered rapidly.

FIG. 3 (c) illustrates that the temperature of the PCR product may be decreased from denaturation temperature (e.g. 95° C.) to a temperature below room temperature (e.g. 10° C.) within 10 seconds.

FIG. 4 illustrates a method 420 for lowering the temperature of a chemical reaction which is carried out in a chemical reaction chip integrated into a chemical reaction module according to one embodiment. The cooling device as described herein may be used to lower the temperature of the chemical reaction. The method 420 may include 403 cooling the chemical reaction chip to a first temperature using the cooling device. The method 420 may further include 404 cooling the chemical reaction chip to a second temperature using the cooling device by initializing an endothermic reaction of the endothermic chemical material in the cooling device.

In one embodiment, the chemical reaction is a biochemical reaction, e.g. a polymerase chain reaction.

According to one embodiment, the initialization of the endothermic reaction of the endothermic chemical material is carried out using a liquid as initial start-up. The liquid may be water, for example.

In one embodiment, the first temperature is a temperature in the range from about 50 to 60° C., and the second temperature is a temperature less than 10° C. The first temperature may be a temperature of approximately 55° C. The second temperature may be a temperature below room temperature.

According to one embodiment, the chemical reaction is a polymerase chain reaction and the first temperature is an annealing temperature. The polymerase chain reaction may be carried out with or without reverse transcription.

The second stage may be carried out for a cooling time of less than 10 seconds.

The method 420 may be also referred to as the two-step passive cooling methods. With the two-step passive cooling method as described herein, there is no need to use any active control instruments for the cooling process. Also this method is applicable to the disposable solution of one time use of nucleic acid sample preparation microsystem. There is also potential to application to the integrated microsystem for nucleic acid based disease diagnostics.

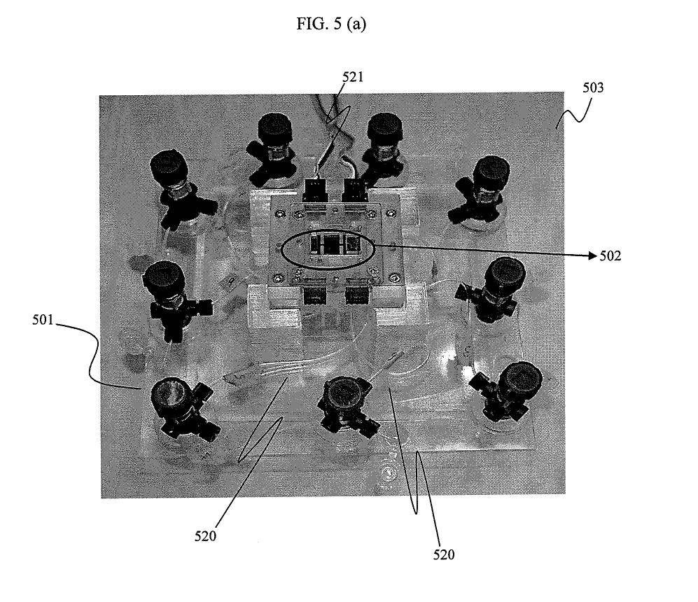

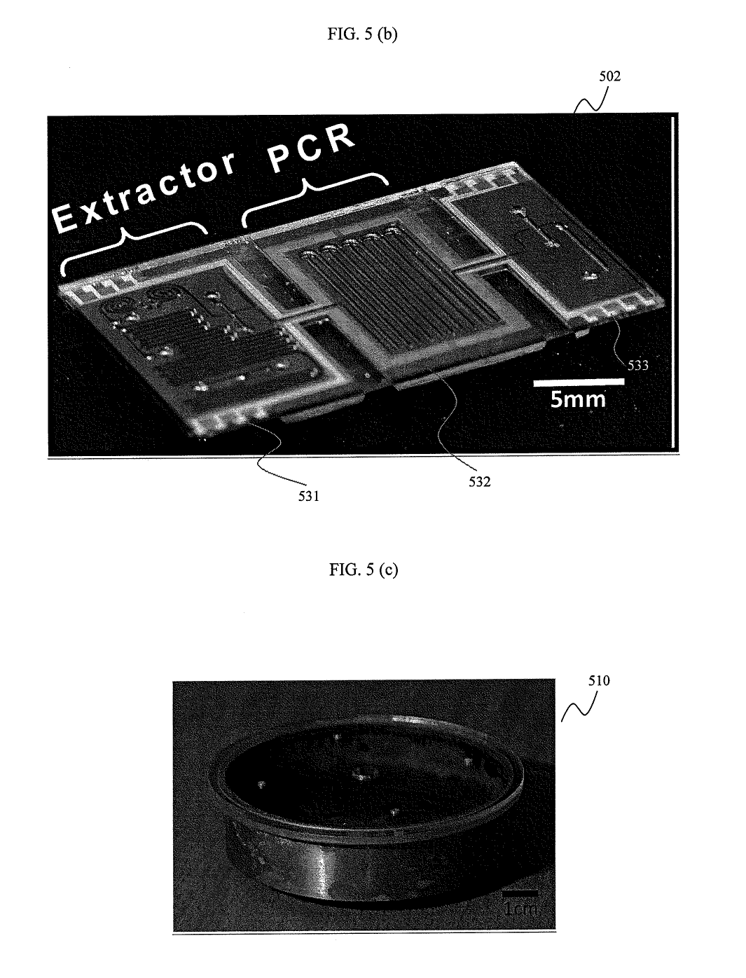

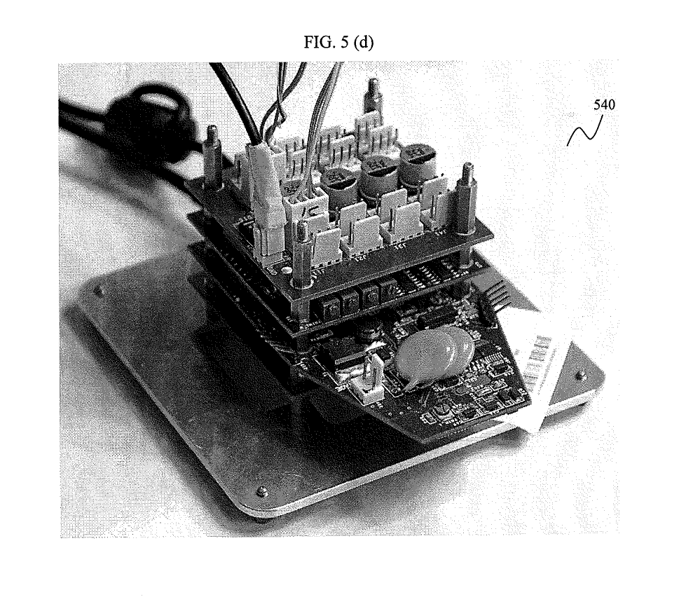

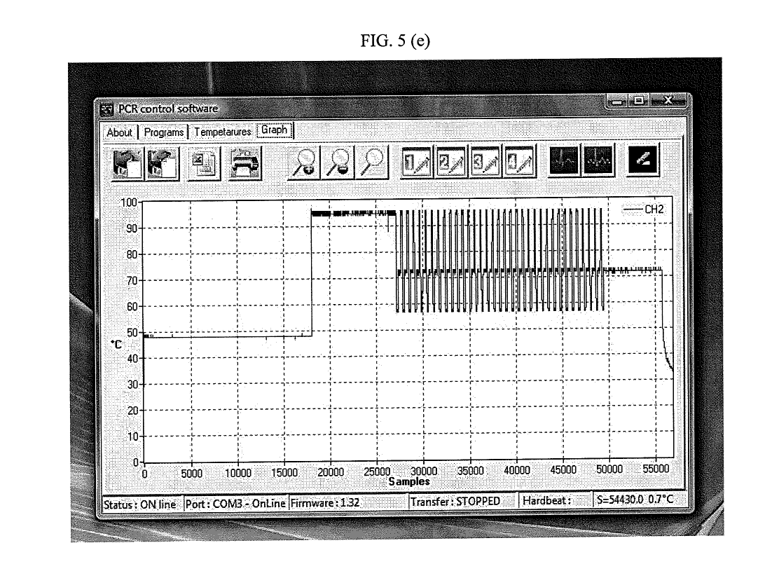

FIG. 5 (a) shows a photo of a module 503 which includes a supporting element 501 and a chemical reaction chip 502 according to one exemplary embodiment. The module 503 may be the same as the module 303 described with reference to FIGS. 3 (a) and (b). The supporting element 501 may be a container.

The supporting element 501 contains fluidic interconnections 520 and electrical interconnections 521. The chemical reaction chip 502 is coupled to the supporting element 501 and may be in fluidic and electrical connection with the supporting element 501. For example, fluidic sample, e.g. blood, may be drawn into microchannels in the chemical reaction chip 502 and the resulted sample may be driven out of the chemical reaction chip upon electronic control via the electrical connections 521. Temperature control and monitoring may also be achieved via a temperature control device which is connected to the module 503 via the electrical connections 521.

FIG. 5 (b) shows an enlarged view of the chemical reaction chip 502 in one exemplary embodiment wherein the chemical reaction is the polymerase chain reaction. In this exemplary embodiment, the chip 502 includes 3 main portions, i.e. the extraction portion 531, the PCR chamber portion 532, and the exit portion 533. Each of the portions 531, 532, and 533 may include microfluidic channels. The extraction portion 531 may be in fluidic connection with the PCR portion 532 via a fluidic channel, and the PCR portion 532 may be in fluidic connection with the exit portion 533 via a fluidic channel.

For example, the working process of the chemical reaction chip 502 may be as follows. Firstly, the lysed blood sample may be injected to the chip 502 through one of the fluidic inlet at extraction portion 531, and the nucleic acid binding may happen on the SiO₂ surface of the extraction portion 531 of the chip 502. Then the microchannel of the chip 502 is washed using washing buffer. After the washing of the microchannel of the chip 502, the binded nucleic acid on the SiO₂ microchannel surface is eluted with the injected water-based elution buffer through the same fluidic inlet at the extraction portion 531. Then eluted nucleic acid sample may be mixed with PCR reagents in the microchannel of the extraction portion 531 and passed to the PCR portion 532. Once mixture of PCR reagents and eluted nucleic acid are filled into the PCR reaction chamber 532, PCR thermal cycling may be conducted. During this thermal cycling, the container of the cooling device 510 may act as a heat sink in order to enhance the cooling efficiency of the PCR reaction chamber 532. Once the PCR reaction has been finished, the temperature of the PCR reaction chamber 532 again is increased up to the denaturation temperature for denaturing the PCR product. At this time, water is injected to the container of the cooling device 510, whose bottom rectangular surface may be physically contacted with top surface of the PCR reaction chamber 532, so that endothermic reaction would be started in order to conduct the rapid cooling. Once the rapid cooling has been finished, the denatured single stranded DNA would be ejected out through the fluidic exit in the exit portion 533.

Assuming the chemical reaction chip 502 is configured to perform a polymerase chain reaction for RNA. In operation, for example, the sample containing the RNA may be injected into the extraction portion 531 via fluidic interconnections 520 in the supporting element 501 of the module 503. For example, RNA may be extracted from the sample in the extraction portion 531 of the chemical reaction chip 502. The extracted RNA may be input into the PCR portion 532 where PCR reaction is carried out such that RNA is amplified. At the end of the PCR thermal cycling period, the PCR product may be heated to a denaturation temperature followed by a super-cooling process to keep the denatured RNA in a single stranded form.

FIG. 5 (c) shows the photo of a cooling device 510 according to an exemplary embodiment. The cooling device 510 may be the same as described herein with reference to FIGS. 2 (a)-(c) and 3 (a)-(b).

The cooling device 510 may be mounted on the module 503 as shown in FIG. 5 (a). The cooling device 510 may be configured to lower the temperature of the chemical reaction carried out in the chemical reaction chip 502. For example, when the chemical reaction is PCR, the cooling device 510 may be configured to lower the temperature of the PCR chamber portion 532 during each cycle of the PCR cycling period from the denaturation temperature to the annealing temperature. After the PCR thermal cycling period, the PCR product may be heated to the denaturation temperature, then an initial start-up material, e.g. water, may be put into the container of the cooling device 510 to initiate an endothermic reaction such that the temperature of the PCR product is lowered from the denaturation temperature to a temperature below room temperature in a fast manner, e.g. within 10 seconds. The PCR product may then be output into the exit portion 533 for further process such as hybridization based detections. The cooling device 510 may include a heat contact element on the outer surface of the base element of the container of the cooling device 510. The heat contact element may be in contact with the chemical reaction chip 502 when the cooling device 510 is mounted on the module 503. For example, the heat contact element of the cooling device 510 may be in contact with the portions 531-532 of the chemical reaction chip 502. For another example, the heat contact element of the cooling device 510 may be in contact of the PCR portion 532 of the chemical reaction chip 502.

FIG. 5 (d) shows a photo of a customized temperature controller 540 which may be connected to the module 503 via the electronic connections 521 according to one exemplary embodiment. The temperature controller 540 may be used to control and monitor the temperature of the chemical reaction carried out in the chemical reaction chip 502. The temperature controller 540 may be further connected to a screen showing the temperature.

FIG. 5 (e) shows a screen shot of the monitoring of the temperature during the PCR thermal cycling period by the temperature controller 540.

After the chemical reaction, e.g. PCR and denaturation of the PCR product, the result may be detected.

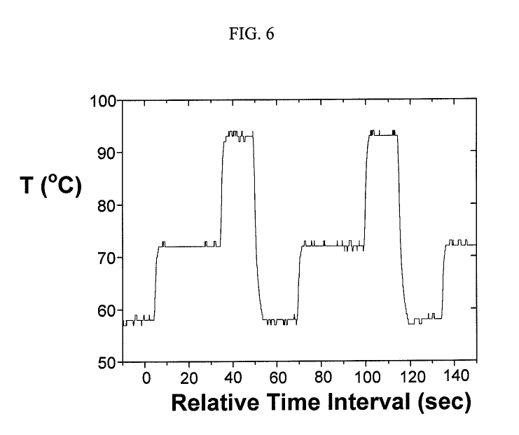

FIG. 6 illustrates the monitoring of temperature changes during a PCR thermal cycling period where PCR is carried out in the assembly as shown in FIG. 3 (b). The cooling device as described herein is used as a heat-sink during the PCR thermal cycling period. As can be seen, the microPCR chamber temperature may be cooled down from 93° C. to 58° C. within 5 seconds using the cooling device as described herein, for the PCR thermal cycling control.

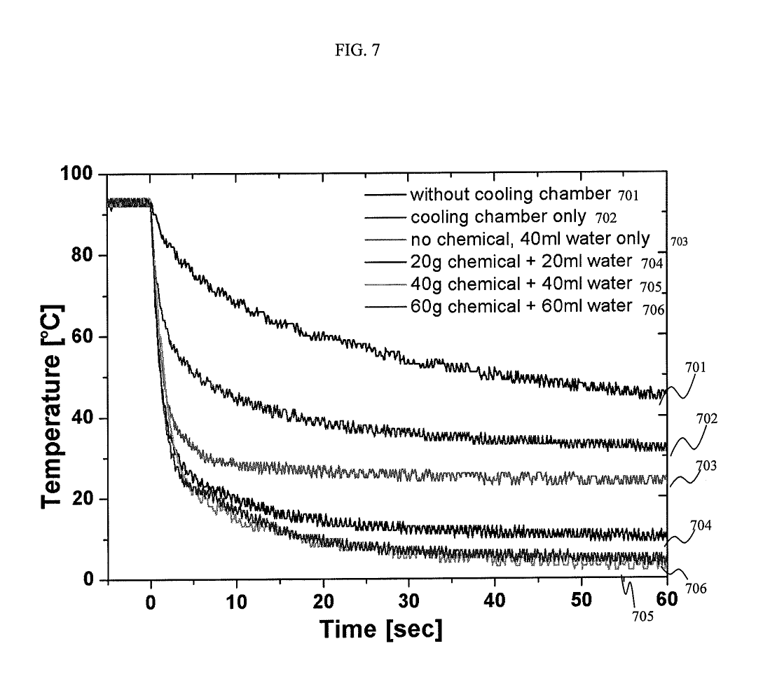

FIG. 7 shows the monitoring of temperature changes after the PCR product is heated to a denaturation temperature under different cooling conditions.

Line 701 shows the temperature change when no cooling device is applied. As can be seen, the temperature dropped to about 70° C. within 10 seconds and drops to about 40° C. within one minute.

Line 702 shows the temperature change where the cooling device is used but no endothermic reaction is initiated. The temperature drops to about 45° C. within 10 seconds and drops to about 30° C. within one minute due to the fact that the cooling device functions as a heat sink and helps to dissipate the heat off the PCR product.

Line 703 shows the temperature change where the cooling device is used without the endothermic chemical material but with 40 ml water added to the container of the cooling device as described herein after the PCR product is heated to the denaturation temperature. As can be seen, the temperature drops to about 30° C. within 10 seconds.

Line 704 shows the temperature change where the cooling device which contains 20 gram of the endothermic chemical material is used and 20 ml water is added to initiate the endothermic reaction after the PCR product is heated to the denaturation temperature. As can be seen, the temperature drops to about 20° C. within 10 seconds and drops to about 10° C. within one minute.

Line 705 shows the temperature change where the cooling device which contains 40 gram of the endothermic chemical material is used and 40 ml water is added to initiate the endothermic reaction after the PCR product is heated to the denaturation temperature. As can be seen, the temperature drops to about 15° C. within 10 seconds and drops to about 5° C. within one minute.

Line 706 shows the temperature change where the cooling device which contains 60 gram of the endothermic chemical material is used and 60 ml water is added to initiate the endothermic reaction after the PCR product is heated to the denaturation temperature. As can be seen, like the line 705, the temperature drops to about 15° C. within 10 seconds and drops to about 5° C. within one minute.

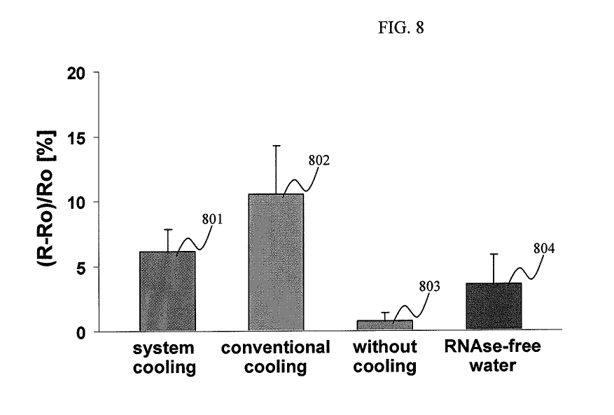

FIG. 8 illustrates the DNA denaturation test with a nanowire biosensor. The nanowire biosensor may be the one as described in G. J. Zhang et al. “Highly sensitive measurements of PNA-DNA hybridization using oxide-etched silicon nanowire biosensors” Biosensors and Bioelectronics 23 (2008) 1701-1707. The Dengue II virus with a concentration of 10³ pfu/ml is extracted from 80 μl of blood in the chemical reaction chip such as in the portion 531 shown in FIG. 5 (b), and then a polymerase chain reaction was performed. DNA of the Dengue II virus is extracted and then amplified by integrated microPCR chip. The Y axis shows the change of resistance of the nanowire, which is an indication of the amount of single stranded DNA in the PCR product. That is, the resistance of the nanowire would change upon the binding of single stranded DNA with the single stranded DNA template attached to the nanowire.

Result 801 is for the PCR product sample which was heated up to the denaturation temperature and then was performed a super cooling process using the cooling device as described herein. Result 802 is for the PCR product sample which is used as a positive control and which is prepared on lab machines (thermocycler), and cooled by dipping the sample in an boiling water and followed by ice water bath. Result 803 is for the PCR product sample which was not heated up to the denaturation temperature so that there was no denaturation steps involved. Result 804 is a negative control where the sample used for this is actually RNAse-free water, which does not include any nucleic acid sample. All samples are detected on a nanowire biosensor functionalized with specific PNA probes.

As shown in the FIG. 8, integrated process of PCR and super cooling by the cooling device described herein shows comparable level of nanowire relative resistance change (801) with the positive control (802). This means that proposed method of two-step passive cooling is able to denaturate the PCR amplicon, which is amplified by microPCR chip. Also comparing with sample 803, it is found that the super-cooling step is very critical for the hybridization based nucleic acid detection.

Overall, embodiments provide a two step passive cooling device including modifying the natural heat sink structure as a container shape in order to get two-step cooling functions such as heat-sink for thermal cycling of PCR/RT-PCR and super-cooling for DNA denaturation. It is experimentally verified that the two-step passive cooling device has a capability to cool down the temperature from a denaturation temperature of PCR cycling (e.g. 93° C.) to an annealing temperature of PCR cycling (e.g. 58° C.) without any additional chemical reaction as well as from the denaturation temperature (e.g. 93° C.) to a temperature lower than room temperature (e.g. less than 10° C.) for the super cooling of DNA denaturation successfully. By adapting the two-step passive cooling device as described herein, the integrated microsystem for nucleic acid based diagnosis of infectious disease would get total disposable solution without any active cooling control units.

It should also be noted that although the invention is described mainly in context of PCR reaction and DNA/RNA denaturation, the cooling device and the assembly as well as the method of lowering temperature of the chemical reaction are not limited to be applied to PCR and DNA denaturation.

The cooling device and the assembly as described herein may be incorporated in an automated and miniaturized “sample-to-answer” microsystem, for early diagnosis of infectious diseases by detecting the viral RNA from a few drops of finger-pricked blood. The integrated system may consist of three major components: 1) viral RNA extraction; 2) nucleic acid amplification; and 3) nucleic acid detection. The system may be packaged as a cartridge, in which different bio-microfluidic components were integrated.

According to the experiments carried out, 1 pfu of Dengue II viral RNA has been successfully extracted from 50 μl of spiked blood by using a silicon chip containing a microchannel coated by silicon-dioxide (SiO2). Integrated microheaters and temperature sensors on the microPCR chip may be fabricated by silicon micromachining. Their thermal cycling performance may be demonstrated with a customized temperature controller. The detection of unpurified PCR product (1 nM) may be carried out in a label-free fashion on a silicon nanowire sensor. This result enabled to eliminate a purification step after amplification of nucleic acid in the integrated system. The integrated microsystem shows the potential of realizing fully automated and miniaturized tools for infectious disease diagnosis.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. A cooling device, comprising:
a container configured as a heat sink, wherein the container is at least partially made from heat conducting material, and
endothermic chemical material, which is contained in the container.

2. The cooling device according to claim 1,
wherein the container comprises a base element and a covering element, which covers the base element.

3. The cooling device according to claim 2,
wherein the base element is a base chamber.

4. The cooling device according to claim 1, further comprising:
a heat contact element on the outer surface of the base element.

5. The cooling device according to claim 4,
wherein the heat contact element is a heat contact plate.

6. The cooling device according to claim 2,
wherein the base element is at least partially made from a heat conducting material.

7. The cooling device according to claim 4,
wherein the heat contact element is at least partially made from a heat conducting material.

8. The cooling device according to claim 4,
wherein the heat contact element is at least partially made from metal.

9. The cooling device according to claim 4,
wherein the heat contact element is at least partially made from at least one material selected from a group of materials consisting of: gold, argent, aluminum, brass, zinc, magnesium, graphite, tungsten, silicon, molybdenum, nickel, iron, steel, platinum, tin, and tantalum.

10. The cooling device according to claim 2,
wherein the base element is at least partially made from metal.

11. The cooling device according to claim 2,
wherein the base element is at least partially made from copper.

12. The cooling device according to claim 2,
wherein the base element is at least partially made from at least one material selected from a group of materials consisting of: gold, argent, aluminum, brass, zinc, magnesium, graphite, tungsten, silicon, molybdenum, nickel, iron, steel, platinum, tin, and tantalum.

13. The cooling device according to claim 2,
wherein the covering element is at least partially made from metal.

14. The cooling device according to claim 2,
wherein the covering element is at least partially made from copper.

15. The cooling device according to claim 2,
wherein the container comprises at least one opening for injecting an initial start-up material into the container.

16. The cooling device according to claim 15,
wherein the at least one opening is arranged on the upper surface of the covering element.

17. Assembly, comprising:
a module configured to perform a chemical reaction, comprising a supporting element and a chemical reaction chip coupled to the supporting element; and
a cooling device according to claim 1 being configured to cool at least a part of the chemical reaction chip;
wherein the biochemical reaction is carried out in the chemical reaction chip.

18. The assembly according to claim 17, wherein the biochemical reaction is a polymerase chain reaction.

19. The assembly according to claim 17, wherein the supporting element contains electrical interconnections and fluidic interconnections.

20. The assembly according to claim 17, wherein the module further comprises a top plate which covers the chemical reaction chip.

21. The assembly according to claim 20,
wherein the top plate comprises an opening.

22. The assembly according to claim 21,
wherein the heat contact element of the cooling device is fed through the opening of the top plate.

23. The assembly according to claim 22,
wherein the heat contact element of the cooling device is fed through the opening of the top plate in such a manner that the heat contact element contacts the top surface of the chemical reaction chip such that thermal conduction between the cooling device and the top surface of the chemical reaction chip is realized.

24. The assembly according to claim 17,
wherein the cooling device is configured as a heat-sink for leading off heat from the module.

25. A method for lowering the temperature of a chemical reaction which is carried out in a chemical reaction chip integrated into a chemical reaction module using the cooling device according to claim 1, comprising:
cooling the chemical reaction chip to a first temperature using the cooling device, cooling the chemical reaction chip to a second temperature using the cooling device by initializing an endothermic reaction of the endothermic chemical material in the cooling device.
